(19) United States
(12) Patent Application Publication
(10) Pub. No.: US 2026/0158198 A1
(43) Pub. Date: Jun. 11, 2026

(54) Method And Apparatus For A Pneumatic Circuit In An Extracorporeal Membrane Oxygenator

(71) Applicant: Breethe, Inc., Halethorpe, MD (US)

(72) Inventors: Bruce Berlage, Danvers, MA (US); Thomas Underwood, Danvers, MA (US); Gregory Herwig, Danvers, MA (US); Brian Wenholz, Danvers, MA (US); Arkadiy Tsitlik, Danvers, MA (US)

(73) Assignee: Breethe, Inc., Halethorpe, MD (US)

(21) Appl. No.: 19/407,984

(22) Filed: Dec. 3, 2025

Related U.S. Application Data
(60) Provisional application No. 63/730,675, filed on Dec. 11, 2024.

(51) Int. Cl. A61M 1/16; A61M 39/22; A61M 39/24

(52) U.S. Cl. CPC A61M 1/1698; A61M 39/24; A61M 2039/226; A61M 2202/0208; A61M 2205/07; A61M 2205/3334; A61M 2205/3341

(57) ABSTRACT

A pneumatic circuit having three inlets and two outlets controls gas flow in the circuit. The circuit allows gas to flow from a second inlet to each of the two outlets controlling flow by a check and a check valve between the second inlet and the second outlet. The first check valve prevents flow from the first inlet to the second outlet. The second check valve prevents flow of gas from the third inlet to the first outlet. A flow meter in communication with the second inlet provides input to a controller controlling gas sources at the first and third inlets when a flow rate is detected from the second inlet. In an extracorporeal membrane oxygenator (ECMO), the second inlet can be connected to wall mounted oxygen. The first inlet can be connected to an oxygen concentrator, and the third inlet is connected to an air pump for providing air.

### CROSS-REFERENCE TO RELATED APPLICATIONS

This application claims the benefit of the filing date of U.S. Provisional Ser. No. 63/730,675 , filed Dec. 11, 2024, the disclosure of which is hereby incorporated herein by reference.

### BACKGROUND

The present invention relates to extracorporeal membrane oxygenation (ECMO), sometimes also referred to as extracorporeal life support (ECLS), and more particularly to a system for sharing oxygen as between different approaches for extracorporeal oxygenation.

Patients awaiting or recovering from a heart or lung transplant and patients having conditions putting them at risk for heart or lung failure may be candidates for ECMO therapy. ECMO therapy is designed to address an excess of carbon dioxide and/or lack of oxygen in the blood where the lungs are not healthy and/or the heart cannot pump enough blood around the body. ECMO is thus an extracorporeal technique which replaces these respiratory and cardiac functions in various situations ranging from support needed while separately treating the underlying causes of cardiac arrest to late-stage treatment for heart or lung failure. It is a therapeutic treatment to provide temporary help when needed. It does not address the underlying condition.

ECMO therapy operates by drawing deoxygenated blood from the body of a patient in a controlled manner to permit oxygenation of the blood and to remove carbon dioxide from the blood. The oxygenated blood is then cycled back into the patient. Generally, ECMO systems include a pump, an oxygenator, an oxygen source, and a control center to monitor and control the process. It may also include a blood warmer to bring the blood back up to the temperature of the body as it is circulated back into the body. Deoxygenated blood is drawn from the body via a cannula and is pumped through an oxygenator where oxygen is infused in the blood and carbon dioxide is removed from the blood. The oxygenated blood is pumped from the oxygenator back into the body, with blood warming in most instances.

A pneumatic circuit provides gas to the oxygenator. The pneumatic circuit can receive a number of separate gas inlets and provide one or more gas outlets. For example, high oxygen gas may be provided to the oxygenator from the pneumatic circuit and used to oxygenate the patients'blood. The pneumatic circuit may have a number of components adding to its complexity and space requirements. Improved designs that reduce space and complexity are desired.

### SUMMARY

A pneumatic circuit is described that includes a plurality of inlets and a pair of outlets. Flow of gasses through the circuit are controlled in part by the positioning of a series of check valves. A default gas source is monitored for the presence of gas entering the circuit. A controller creates control signals for controlling other gas sources connected to one or more inlets to the circuit.

According to embodiments described in detail below, a pneumatic circuit for a blood oxygenator includes a first inlet of a first gas source, a second inlet of a second gas source, and a third inlet of a third gas source. A first gas outlet is in fluid communication with the first inlet and connected to the blood oxygenator and a second gas outlet is in fluid communication with the third inlet and connected to the blood oxygenator. A first check valve is placed between the first inlet and the second outlet preventing flow of the first gas from the first inlet to the second outlet. A second check valve is placed between the third inlet and the first outlet preventing flow of the third gas from the third inlet to the first outlet. The first and second outlets are in fluid communication with the second inlet and provide oxygenated gas to the blood oxygenator. The first and second check valve are configured to allow gas flow from the second inlet to the first outlet and the second outlet for provision to the blood oxygenator.

A solenoid valve is positioned between the first gas inlet and the first outlet for selectively preventing flow of gas from the first gas inlet to the first outlet. A flow meter placed between the second gas inlet and the first second check valves measures the flow of gas from the second gas inlet and the first outlet and the second outlet. The flow meter is in communication with a controller; the controller can provide a control signal to the solenoid valve in response to an output of the flow meter. Additionally, the controller can provide a control signal to close the solenoid valve in response to the output of the flow meter indicating a flow of gas from the second gas inlet through the flow meter, or a control signal to open the solenoid valve in response to the output of the flow meter indicating that no flow of gas exists between the second gas inlet and the flow meter.

An air pump to provide air flow at the third gas inlet, wherein the controller provides a control signal to turn off the air pump in response to the output of the flow meter indicating a flow of gas from the second gas inlet through the flow meter, or a control signal to turn on the air pump in response to the output of the flow meter indicating that no flow of gas exists between the second gas inlet and the flow meter.

A second flow meter placed between the third gas inlet and the second outlet measures a gas flow rate from the third gas inlet and the second outlet. The controller is in communication with the second flow meter and the air pump and controls an air output of the air pump in response to the measured gas flow rate at the second flow meter.

In another aspect, the pneumatic circuit of claim includes a pressure regulator for controlling a gas pressure at the second gas inlet and a pressure relief device for maintaining a maximum pressure at the second gas inlet. A balancing orifice situated between the first check valve and the second check valve to balance a flow of gas from the second gas inlet between the first outlet and the second outlet. a third check valve is placed between the first check valve and the first gas inlet that prevents gas flow from the second gas inlet to the first gas inlet.

According to some embodiments, the first gas source is an oxygen concentrator, the second gas source is a wall mounted oxygen gas outlet or a pressurized cylinder containing oxygen gas, and the third gas source is an air pump drawing ambient air into the third inlet. The circuit can be configured to operate in a default condition where a gas source for the circuit is from the second gas source. If gas is provided to the circuit via the second gas inlet of the second gas source the default condition includes preventing gas from the first gas inlet and the third gas inlet from reaching the first or second gas outlet.

A method for controlling gas flow in a pneumatic circuit for a blood oxygenator includes directing flow from a plurality of gas sources to a first outlet and a second outlet connected to the blood oxygenator, preventing flow from a first gas source to the second outlet by placing a first check valve between the first gas source and the second outlet, preventing flow from a third gas source to the first outlet by placing a second check valve between the third gas source and the first outlet, and allowing gas flow from a second gas source to the first outlet and the second outlet via the first check valve and the second check valve. The introduction of gas from the first gas source and the introduction of a gas from the third gas source can be controlled based on a detection of a gas flow rate from the second gas source. The introduction of gas from the first gas source can be stopped when gas flow is detected from the second gas source and the introduction of gas from the third gas source is stopped when gas flow is detected from the second gas source. the introduction of gas from the first gas source can be achieved by closing a solenoid valve between the first gas source and the first outlet, and stopping the introduction of gas from the third gas source can be achieved by controlling the operation of an air pump connected to the second outlet.

### BRIEF DESCRIPTION OF THE DRAWINGS

FIG. 1 is a block diagram of a pneumatic circuit according to aspects of this disclosure.

FIG. 2 is a block diagram of the pneumatic circuit of FIG. 1 illustrating control features of the circuit according to aspects of this disclosure.

FIG. 3 is a diagram of a default state for a pneumatic circuit according to aspects of this disclosure.

FIG. 4 is a diagram of multiple states of a pneumatic circuit according to aspects of this disclosure.

FIG. 5 is a process flow diagram for controlling a pneumatic circuit according to aspects of this disclosure.

FIG. 6 is a block diagram of a conventional pneumatic circuit.

FIG. 7 is an illustration of a blood oxygenator including a pneumatic circuit according to aspects of the embodiments of this disclosure.

FIG. 8 is a block diagram of a pneumatic circuit according to aspects of this disclosure.

### DETAILED DESCRIPTION

Pneumatic circuits are devices or assemblies that control and transport a flow of one or more gases. A pneumatic circuits includes at least one inlet for receiving gas from a first gas source. Flow and direction of the gas received at the inlet(s) is controlled through the circuit and exits the circuit via one or more outlets. Sensors and microcontrollers can be used to sense the state of the gas(es) in the circuit and to direct their flow through the circuit. The design of the circuit can be complex and contain many components. The higher number of components needed typically results in increased size of the circuit enclosure. In portable applications it is desirable to reduce the size and weight of the circuit to make the device containing the pneumatic circuit easier to transport.

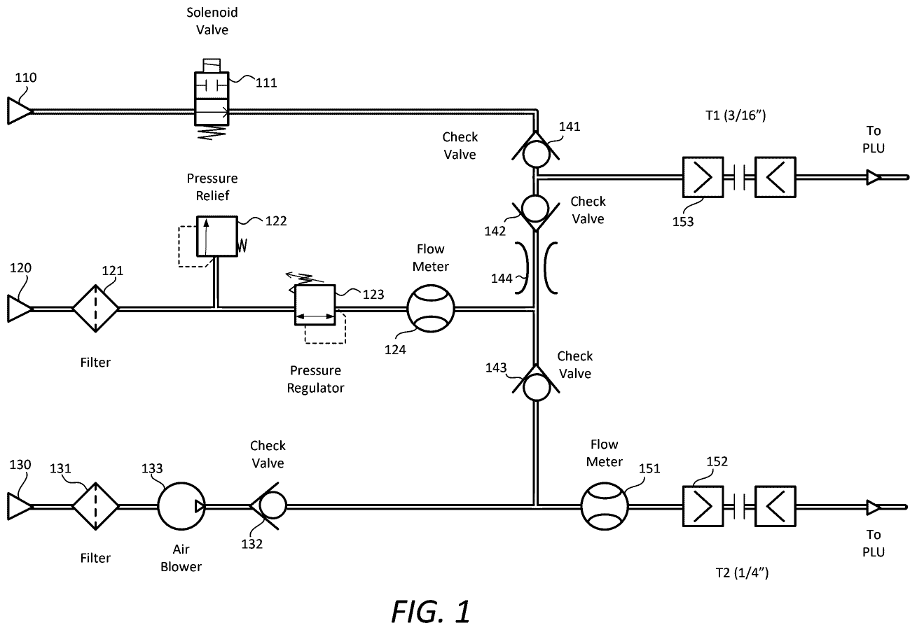

FIG. 1 is a block diagram of a pneumatic circuit according to aspects of embodiments described in this disclosure. The pneumatic circuit has three inlets, a first inlet 110, a second inlet 120 and a third inlet 130. According to one example, the first inlet 110 can be connected to an oxygen concentrator. The second inlet 120 can be connected to a wall outlet that dispenses oxygen gas or alternatively to a pressurized cylinder containing oxygen gas. The third inlet 130 can provide ambient air that is drawn into the third inlet 130 by an air pump 133 that directs air flow in through inlet 130 and a filter 131.

As stated above, first inlet 110 may be connected to an oxygen concentrator. An oxygen concentrator accepts atmospheric air and passes the air through filters to filter out the nitrogen in the air leaving behind concentrated oxygen that passes through. The oxygen is directed to a tank for storage. The stored oxygen may be provided by a tube and cannula to a patient's nose, providing oxygen rich air to the patient. In the pneumatic circuit of FIG. 1, the first inlet 110 may provide oxygen rich gas to first inlet 110. The flow of the oxygen rich gas to the circuit may be controlled by a solenoid valve 111, which selectively allows gas from the first inlet 110 to enter the circuit. When solenoid valve 111 is open, gas may enter the circuit through first inlet 110, pass through check valve 141 and exit the circuit through the first inlet 153. Check valve 142 prevents gas entering through the first inlet 110 from reaching the second outlet 152.

Third inlet 130 receives ambient air through a pressure differential created by air pump 133. The air passes through filter 131 and check valve 132, passing through flow meter 151 and out second outlet 152. Check valve 143 is situated to prevent flow of air from the third inlet 130 to the first outlet 153.

Second inlet 120 provides gas to the circuit through filter 121. In one example, the gas entering the circuit by second inlet 120 represents a default state of the circuit where second inlet 120 is the primary source of gas to the circuit. A pressure regulator 123 controls the amount of gas flowing into the circuit from second inlet 120. A pressure relief device 122 enforces a maximum gas pressure of gas at the second inlet 120. A flow meter 124 monitors the flow of gas from the second inlet 120. The gas from second inlet 120 enters a balancing orifice 144 that equalizes the flow of gas between second inlet 120 to first outlet 153 via check valve 142 and the flow of gas from second inlet 120 to second outlet 152 via check valve 143. Check valve 141 prevents gas from second inlet 120 from backing up into first inlet 110 while check valve 132 prevents gas from second inlet 120 from backing up into third inlet 130.

Outlets 153, 152 may be connected to a pump lung unit (PLU) of a blood oxygenator in an ECMO device. As a default, the pneumatic circuit accepts wall oxygen as a default through second inlet 120. When wall oxygen is not available, the pneumatic circuit will switch to direct oxygenated gas from an oxygen concentrator associated with the ECMO unit at first inlet 110 through first outlet 153. Simultaneously, air is directed to the second outlet 152 via third inlet 130. Flow meter 151 monitors the air flow through second outlet 152, which may be controlled through air pump 133. Check valve 143 prevents air from third inlet 130 from reaching first outlet 153, while check valve 142 prevents oxygen rich gas from first inlet 110 from reaching second outlet 152.

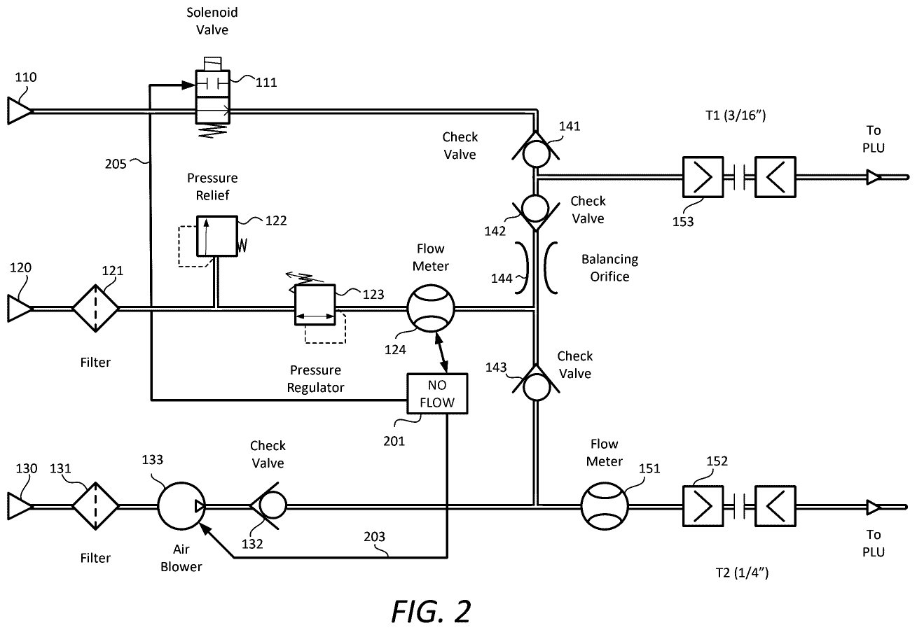

FIG. 2 is a block diagram of the pneumatic circuit described above with regard to FIG. 1 illustrating a control scheme for controlling the operating state of the pneumatic circuit. The circuit of FIG. 2 is configured to define a default state where gas is received through second inlet 120. When the primary source of gas exists, gas sources associated with first inlet 110 and third inlet 130 are not used.

To implement this configuration, flow meter 124 connected to second inlet 120 monitors the gas flow level through the flow meter 124. Flow meter 124 is in communication with a controller 201, which is communicatively connected to solenoid valve 111 associated with the first inlet 110 and air pump 133 associated with third inlet 130.

When gas (e.g., oxygen from a wall connection) is present and flowing from second inlet 120, flow meter 124 detects the presence of the gas and generates a signal to the controller 201 indicative of the state of gas flow from second inlet 120. If no gas presence is detected at second inlet 120, the flow meter will read zero gas flow and generate a signal to the controller 201 that no gas is present at second inlet 120.

Upon receiving the signal that no gas is present at the second inlet 120, the controller 201 generates and communicates a control signal 205 to solenoid valve 111 to open and allow gas to begin flowing from first inlet 110 through check valve 141 and out first inlet 110. In addition, the controller 201 generates a control signal 203 to air pump 133 to turn on and begin drawing ambient air through third inlet 130. The air travels through filter 131, check valve 132, and flow meter 151 then exiting through second outlet 152.

When flow meter 124 detects the presence of gas flow through second inlet 120, the circuit will act to disable the gas sources at first inlet 110 and third inlet 130. Flow meter 124 communicates to controller 201 that gas is present. In response to the communication, controller sends a control signal 205 to solenoid valve 111 to close and prevent gas from entering the circuit through first inlet 110. In addition, controller 201 generates and sends control signal 203 to air pump 133 to turn off the air pump 133 and stop air from entering third inlet 130.

The circuit of FIG. 2 controls the state of the pneumatic circuit by monitoring the default condition with respect to the presence of gas at flow meter 124. To enable or disable the entry of gasses from first inlet 110 and third inlet 130, controller 204 provides control signals 203, 205 to operate air pump 133 and solenoid valve 111, respectively. The directing of gases throughout the circuit is controlled using check valves 141, 142, 143, 132. Check valves 142 and 143 allow gas from second inlet 120 to flow to both first outlet 153 and second outlet 152 in a default state where the source gas is provided via second inlet 120. When gas is provided through first inlet 110 and third inlet 130, check valves 142, 143 isolate the first inlet 110 from the second outlet 152 and the third inlet 130 from the first outlet 153. The use of check valves to control flow through the circuit eliminates the need for complex solenoid valves used in conventional solutions. This reduces complexity and reduces spatial requirements for the pneumatic circuit.

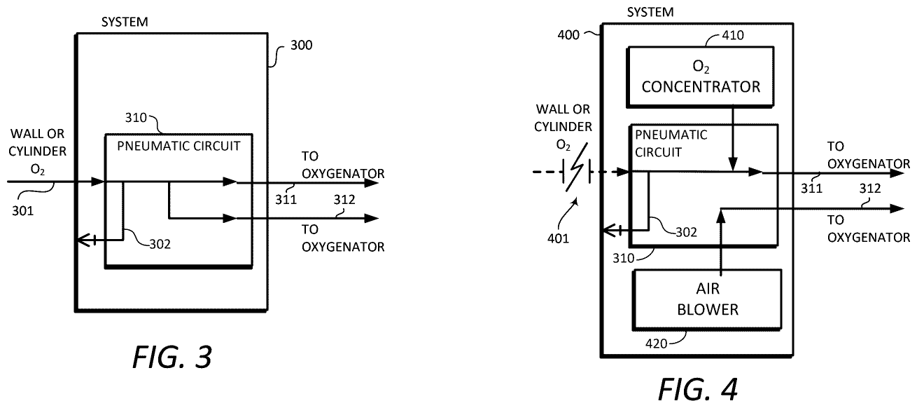

FIG. 3 is a block diagram of a default state for a pneumatic circuit according to an embodiment of this disclosure. The system 300 containing the pneumatic circuit 310 receives an input from a connection 301 to wall oxygen or pressurized cylinder containing oxygen gas. The pneumatic circuit 301 includes a pressure relief device 302 for defining a maximum pressure for pneumatic circuit 301. Pneumatic circuit 310 includes a first outlet 311 and second outlet 312 that are connected to an oxygenator, such as an ECMO.

FIG. 4 is a block diagram of a system 400 operating in a secondary state where wall oxygen is not available 401. Pneumatic circuit 310 receives a first input from oxygen concentrator 410 and a second input from air pump 420. The oxygen concentrator 410 provides oxygen rich gas to a first outlet 311. Air pump 420 provides ambient air to the pneumatic circuit 310 and outputs the air flow through the second outlet 312.

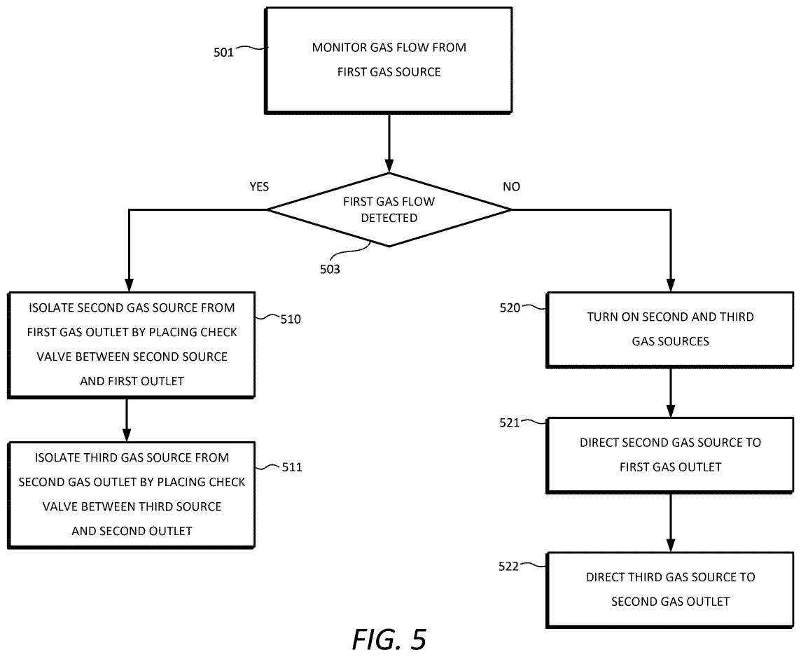

FIG. 5 is a process flow diagram for managing states of a pneumatic circuit in accordance with aspects of embodiments of this disclosure. In a pneumatic circuit, such as the circuit shown and described in FIG. 1 and FIG. 2, gas flow is monitored from a first gas source 501. By way of non-limiting example, the first gas source may be a wall oxygen outlet for providing oxygen rich gas to the circuit. The gas flow is monitored to detect a flow of the first gas 503. If flow is detected, a second gas inlet is isolated from a first outlet by placing a check valve between the second gas inlet and the first outlet 510. A third gas inlet is isolated from the second gas outlet by placing a check valve between the third gas source and the second outlet 511.

If no gas flow is detected 503, the second and third gas sources are turned on 520. The second gas source is directed to the first outlet 521 and the third gas source is directed to the second outlet 522. The process flow may be carried out by monitoring the first gas flow in a flow meter that is in communication with a controller. The controller can generate control signals for the second and third gas sources based on the flow meter's detection of gas flow of the first gas. The controller's role may be limited to turning the secondary sources on or off based on the detection of the first gas flow. Internal flows of gases in the circuit are controlled through the placement of check valves to direct flow to the first and second outlets.

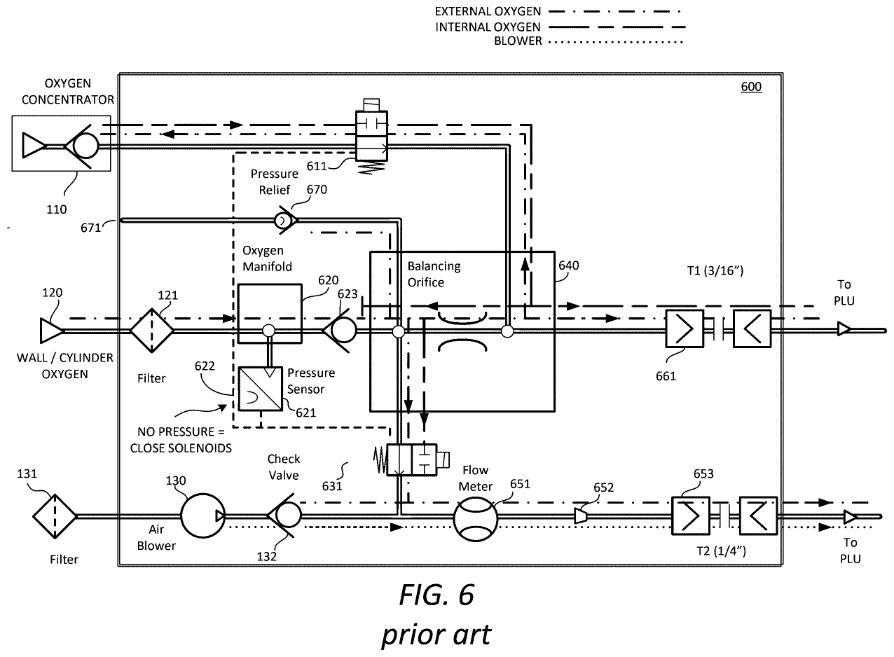

Referring to FIG. 6, a conventional pneumatic circuit is shown. Throughout this description the figures have referred to a pneumatic circuit that supplies oxygenated gas to an extracorporeal membrane oxygenation machine. However, the concepts are applicable to other uses, and the scope of this description and the accompanying claims should not be limited to any particular use case based on the description, which is provided merely by way of example.

Pneumatic circuit 600 receives one or more gas sources such as oxygen concentrator inlet 110, oxygen gas from a wall outlet or pressurized cylinder inlet 120, and atmospheric air 131. The gas may pass through a filter 121, 131 as it enters the pneumatic circuit 600. In the example described with respect to pneumatic circuit 600, the circuit may be used to provide oxygenated gas to a blood oxygenator, such as used in an ECMO. The circuit 600 includes three inlets a first inlet connected to an oxygen concentrator inlet 110, a second inlet connected to an oxygen connection via a wall or cylinder inlet 120, and a third inlet connected to atmospheric air via filter 131 drawn into the circuit by blower inlet 130. The pneumatic circuit 600 is configured to default to an oxygen source entering the circuit via wall/cylinder inlet 120. The gas entering via wall/cylinder inlet 120 passes through filter 121 and enters oxygen manifold 620. Attached to the oxygen manifold is a pressure sensor 621. The gas exits the oxygen manifold 620 passing through check valve 623. Check valve 623 restricts flow in one direction, from the oxygen manifold 620 to balancing orifice 640. Check valve prevents gases from moving from the balancing orifice 640 back into the oxygen manifold 620. The balancing orifice 640 balances gas pressures between the first outlet 661 and second outlet 653.

Gas pressure within the balancing orifice 640 is applied to a pressure relief valve 670 that is connected to a vent 671 leading out of the pneumatic circuit 600. Auxiliary gas sources via oxygen concentrator inlet 110 and blower inlet 130 are blocked from entering the balancing orifice by 2-way solenoid valve 611 and 2-way solenoid valve 631, respectively. 2-way solenoid valves 611, 631 are open and closed in response to a signal provided by pressure sensor 621. If the pressure sensor 621 indicates that a measure pressure of gas from wall/cylinder inlet 120 is greater than an acceptable threshold, then the 2-way solenoid valve 611 will be closed and solenoid valve 631 will be opened to distribute gas from the inlet 120 to the outlets 661 and 653. Oxygenated gas from the wall/cylinder inlet 120 passes out of the balancing orifice 640 to first outlet 661 to a pump lung unit (PLU). The oxygenated gas passes through 2-way solenoid valve 631 through flow meter 651 and pipe diameter reducer 652 and exits the circuit via second outlet 653 to the PLU.

The pneumatic circuit 600 may be designed as part of a portable ECMO unit that allows an ambulatory patient to move about freely with the ECMO machine at their side. In these cases, the pneumatic circuit 600 is disconnected from the wall inlet 120. When this occurs, the pressure sensor 621 will detect the disconnection as a low pressure reading in the oxygen manifold 120. In response to the low-pressure condition, 2-way solenoid valve switches to close the pathway between the balancing orifice 640 and the second outlet 653. Air from blower inlet 130 passes through check valve 132, flow meter 651, pipe diameter reducer 652 and exits the circuit via second outlet 653. Meanwhile, the no pressure condition creates a signal to 2-way solenoid valve 611 to open the path allowing oxygen concentrator inlet 110 to introduce oxygen rich gas to the balancing orifice 640. The oxygen rich gas exits the pneumatic circuit 600 via the first outlet 661. Pressure in the balancing orifice 640 is limited by pressure relief valve 670.

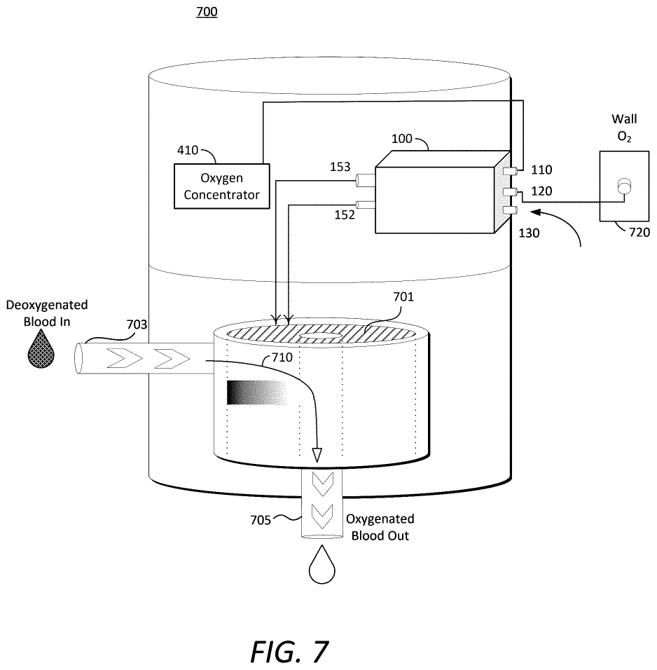

Referring now to FIG. 7, a blood oxygenator 700 is shown. Blood oxygenator 700 is a device that is outside the body of a patient, which receives the patient's deoxygenated blood and infuses oxygen into the blood and returns the oxygenated blood to the patient. The blood oxygenator includes an oxygen concentrator 410. Oxygen concentrator 410 includes a molecular sieve that separates nitrogen from room air and allows oxygen to pass through. The oxygen is stored in a tank within the oxygen concentrator 410. Oxygen concentrator 410 can provide oxygen rich gas to a first inlet 110 of the pneumatic circuit 100. A second inlet 120 of the pneumatic circuit 100 may be connected to a wall mounted oxygen outlet 720. Ambient air may be provided to a third inlet 130 of the pneumatic circuit 100.

The pneumatic circuit 100 includes a first outlet 153 and a second outlet 152 that provides oxygenated gas to a selectively permeable membrane 701. The deoxygenated blood from the patient enters the blood oxygenator 700 through inlet 703. Inlet 703 directs the deoxygenated blood to the outer surface of the selectively permeable membrane 701. The selectively permeable membrane may include a number of hollow fibers that facilitate the flow of oxygenated gas through the selectively permeable membrane 701. The deoxygenated blood is directed across the cross section of the selectively permeable membrane 701 to an outlet passage 705. As the blood passes through the selectively permeable membrane 701 the blood absorbs oxygen through the surfaces of the selectively permeable membrane 701 due to the relative difference in oxygen concentration between the oxygenated gas from the pneumatic circuit 100 and the blood. The exiting blood is oxygenated and provided back to the patient to provide oxygen to vital system in the patient.

As described above with respect to FIG. 1 and FIG. 2, the pneumatic circuit may be configured to operate in a default mode, where the source gas supplied to the circuit is wall oxygen 720 introduced through inlet 120. The pneumatic circuit 100 may disconnect inputs from the first inlet 110 and the third inlet 130 when the default gas source is available. Secondary sources of gas from the oxygen concentrator 410 may be enabled when there is an absence of wall oxygen 720.

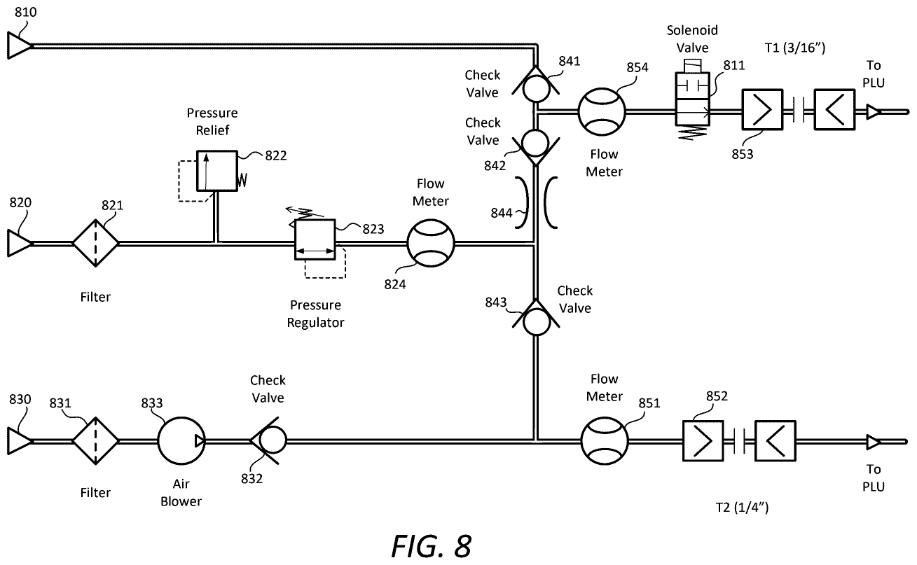

FIG. 8 is a block diagram of a pneumatic circuit according to aspects of an embodiment described in this disclosure. The pneumatic circuit has three inlets, a first inlet 810, a second inlet 820 and a third inlet 830. According to one example, the first inlet 810 can be connected to an oxygen concentrator. The second inlet 820 can be connected to a wall outlet that dispenses oxygen gas or alternatively to a pressurized cylinder containing oxygen gas. The third inlet 830 can provide ambient air that is drawn into the third inlet 830 by an air pump 833 that directs air flow in through inlet 830 and a filter 831.

As stated above, first inlet 810 may be connected to an oxygen concentrator. An oxygen concentrator accepts atmospheric air and passes the air through molecular sieve to separate out the nitrogen in the air leaving behind concentrated oxygen that passes through. The oxygen is directed to a tank for storage. The stored oxygen may be provided by a tube and cannula to a patient's nose, providing oxygen rich air to the patient. In the pneumatic circuit of FIG. 8, the first inlet 810 may provide oxygen rich gas to first inlet 810. The flow of the oxygen rich gas to the circuit may be controlled by a solenoid valve 811, which selectively allows gas from the first inlet 810 and second inlet 820 to enter the circuit. When solenoid valve 811 is open, gas may enter the circuit through first inlet 810, pass through check valve 841 and exit the circuit through the first inlet 853. Check valve 842 prevents gas entering through the first inlet 810 from reaching the second outlet 852.

Third inlet 830 receives ambient air through a pressure differential created by air pump 833. The air passes through filter 831 and check valve 832, passing through flow meter 851 and out second outlet 852. Check valve 843 is situated to prevent flow of air from the third inlet 830 to the first outlet 853.

Second inlet 820 provides gas to the circuit through filter 821. In one example, the gas entering the circuit by second inlet 820 represents a default state of the circuit where second inlet 820 is the primary source of gas to the circuit. A pressure regulator 823 controls the amount of gas flowing into the circuit from second inlet 820. A pressure relief device 822 enforces a maximum gas pressure of gas at the second inlet 820. A flow meter 824 monitors the flow of gas from the second inlet 820. The gas from second inlet 820 enters a balancing orifice 844 that equalizes the flow of gas between second inlet 820 to first outlet 853 via check valve 842 and the flow of gas from second inlet 820 to second outlet 852 via check valve 843. Check valve 841 prevents gas from second inlet 820 from backing up into first inlet 810 while check valve 832 prevents gas from second inlet 820 from backing up into third inlet 830.

Outlets 853, 852 may be connected to a pump lung unit (PLU) of a blood oxygenator in an ECMO device. As a default, the pneumatic circuit accepts wall oxygen as a default through second inlet 820. When wall oxygen is not available, the pneumatic circuit will switch to direct oxygenated gas from an oxygen concentrator associated with the ECMO unit at first inlet 810 through first outlet 853. Simultaneously, air is directed to the second outlet 852 via third inlet 130. Flow meter 851 monitors the air flow through second outlet 852, which may be controlled through air pump 833. Another flow meter 854 is associated with the first outlet 853 that may monitor the gas flow through the first outlet 853 to control the introduction of gas through first inlet 810 and/or second inlet 820 by providing measurement of the flow through flow meter 854 to a controller. Check valve 843 prevents air from third inlet 830 from reaching first outlet 853, while check valve 842 prevents oxygen rich gas from first inlet 810 from reaching second outlet 852.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A pneumatic circuit for a blood oxygenator comprising:
a first inlet of a first gas source (110);
a second inlet of a second gas source (120);
a third inlet of a third gas source (130);
a first gas outlet (153) in fluid communication with the first inlet (110) and connected to the blood oxygenator;
a second gas outlet (152) in fluid communication with the third inlet (130) and connected to the blood oxygenator;
a first check valve (142) placed between the first inlet (110) and the second outlet (152), the first check valve (142) preventing flow of the first gas from the first inlet (110) to the second outlet (152); and
a second check valve (143) placed between the third inlet (130) and the first outlet (110), the second check valve (143) preventing flow of the third gas from the third inlet (130) to the first outlet (153).

2. The pneumatic circuit of claim 1, further comprising:
the first check valve (142) and the second check valve (143) in fluid communication with the second inlet (120), the first check valve (142) and the second check valve (143) configured to allow gas flow from the second inlet (120) to the first outlet (153) and the second outlet (152) for provision to the blood oxygenator.

3. The pneumatic circuit of claim 1, further comprising a solenoid valve (111) positioned between the first gas inlet (110) and the first outlet (153) for selectively preventing flow of gas from the first gas inlet (110) to the first outlet (153).

4. The pneumatic circuit of claim 3, further comprising a flow meter (124) placed between the second gas inlet (120) and the first check valve (142) and the second check valve (143) to measure the flow of gas from the second gas inlet (120) and the first outlet (153) and the second outlet (152).

5. The pneumatic circuit of claim 4, further comprising:
a controller (201) in communication with the flow meter (124), the controller (201) providing a control signal to the solenoid valve (111) in response to an output of the flow meter (124).

6. The pneumatic circuit of claim 5, wherein the controller (201) provides a control signal to close the solenoid valve (111) in response to the output of the flow meter (124) indicating a flow of gas from the second gas inlet (120) through the flow meter (124), or a control signal to open the solenoid valve (111) in response to the output of the flow meter (124) indicating that no flow of gas exists between the second gas inlet (120) and the flow meter (124).

7. The pneumatic circuit of claim 6, further comprising:
an air pump (133) to provide air flow at the third gas inlet (130), wherein the controller (201) provides a control signal to turn off the air pump (133) in response to the output of the flow meter (124) indicating a flow of gas from the second gas inlet (120) through the flow meter (124), or a control signal to turn on the air pump (133) in response to the output of the flow meter (124) indicating that no flow of gas exists between the second gas inlet (120) and the flow meter (124).

8. The pneumatic circuit of claim 7, further comprising:
a second flow meter (151) placed between the third gas inlet (130) and the second outlet (152), the second flow meter (151) measuring a gas flow rate from the third gas inlet (130) and the second outlet (152).

9. The pneumatic circuit of claim 8, wherein the controller is in communication with the second flow meter (151) and the air pump (133) and controls an air output of the air pump (133) in response to the measured gas flow rate at the second flow meter (151).

10. The pneumatic circuit of claim 3, further comprising:
a third flow meter (854) placed between the third gas inlet (830) and the first outlet (853), the third flow meter (854) measuring a gas flow rate from the first or second gas inlet (810, 820) and the first outlet (853).

11. The pneumatic circuit of claim 10, wherein the controller is in communication with the third flow meter (854) and the pressure regulator 823 and controls a gas output of the first or second inlet (810, 820) in response to the measured gas flow rate at the third flow meter (854).

12. The pneumatic circuit of claim 1 further comprising:
a pressure regulator (123) for controlling a gas pressure at the second gas inlet (120); and
a pressure relief device (122) for maintaining a maximum pressure at the second gas inlet (120).

13. The pneumatic circuit of claim 1, further comprising:
a balancing orifice (144) situated between the first check valve (142) and the second check valve (143) to balance a flow of gas from the second gas inlet (120) between the first outlet (153) and the second outlet (152) for providing the gas to the blood oxygenator.

14. The pneumatic circuit of claim 1, further comprising a third check valve (141) between the first check valve (142) and the first gas inlet (110) configured to prevent gas flow from the second gas inlet (120) to the first gas inlet (110).

15. The pneumatic circuit of claim 1, wherein the first gas source is an oxygen concentrator of the blood oxygenator.

16. The circuit of claim 15, wherein the second gas source is a wall mounted oxygen gas outlet, or a pressurized cylinder containing oxygen gas.

17. The circuit of claim 16, wherein the circuit is configured to operate in a default condition where a gas source for the circuit is from the second gas source, entering the circuit from the second gas source (120) and being directed to the blood oxygenator via the first outlet and the second outlet.

18. A method of controlling gas flow in a pneumatic circuit of a blood oxygenator, comprising:
directing flow from a plurality of gas sources to a first outlet (153) and a second outlet (152), the first outlet and the second outlet connected to the blood oxygenator;
preventing flow from a first gas source (110) to the second outlet (152) by placing a first check valve (142) between the first gas source (110) and the second outlet (152);
preventing flow from a third gas source (130) to the first outlet (153) by placing a second check valve (143) between the third gas source (130) and the first outlet (153); and
allowing gas flow from a second gas source (120) to the first outlet (153) and the second outlet (152) via the first check valve (142) and the second check valve (143) and providing the gas to the blood oxygenator.

19. The method of claim 18, further comprising:
controlling an introduction of gas from the first gas source (110) and the introduction of a gas from the third gas source (130) based on a detection of a gas flow rate from the second gas source (120).

20. The method of claim 18 further comprising:
stopping the introduction of gas from the first gas source (110) when gas flow is detected from the second gas source (120) and stopping the introduction of gas from the third gas source when gas flow is detected from the second gas source.
